SEQUENCE LISTING (11 sequences)

SEQ ID NO: 1 - protein, 5 aa, Artificial Sequence
  synthetic peptide
IKVAV

SEQ ID NO: 2 - protein, 28 aa, Artificial Sequence
  synthetic peptide
KIAALKAKIAALKAEIAALEAENAALEA

SEQ ID NO: 3 - protein, 28 aa, Artificial Sequence
  synthetic peptide
KIAALKAKNAALKAEIAALEAEIAALEA

SEQ ID NO: 4 - protein, 28 aa, Artificial Sequence
  synthetic peptide
KIAALKAKIAALKAEIAALEWENAALEA

SEQ ID NO: 5 - protein, 28 aa, Artificial Sequence
  synthetic peptide
KIAALKAKNAALKAEIAALEWEIAALEA

SEQ ID NO: 6 - protein, 28 aa, Artificial Sequence
  synthetic peptide
KIQQLKQKIQQLKQEIQQLEQENQQLEQ

SEQ ID NO: 7 - protein, 28 aa, Artificial Sequence
  synthetic peptide
KIQQLKQKNQQLKQEIQQLEQEIQQLEQ

SEQ ID NO: 8 - protein, 28 aa, Artificial Sequence
  synthetic peptide
KIAALKAKIAALKAEIAALEWENAALEK

SEQ ID NO: 9 - protein, 28 aa, Artificial Sequence
  synthetic peptide
KIAALKAKIAALKAEIAALEWENAALEK

SEQ ID NO: 10 - protein, 28 aa, Artificial Sequence
  synthetic peptide
KIAALKQKIAALKQEIAALEQENAALEQ

SEQ ID NO: 11 - protein, 28 aa, Artificial Sequence
  synthetic peptide
KIAALKQKNAALKQEIAALEQEIAALEQ

(19) United States
(12) Patent Application Publication
(10) Pub. No.: US 2011/0189284 A1
(43) Pub. Date: Aug. 4, 2011

(54) Peptide Fibres

(75) Inventors: Derek Woolfson, Bristol (GB); Edgardo Abelardo, Bristol (GB); Eleanor Banwell, Saitama (JP)

(73) Assignee: UNIVERSITY OF BRISTOL, Bristol (GB)

(21) Appl. No.: 12/974,561

(22) Filed: Dec. 21, 2010

Related U.S. Application Data
(60) Provisional application No. 61/288,702, filed on Dec. 21, 2009.

(51) Int. Cl. A61K 9/10; C07K 2/00; C07K 14/00; C07K 19/00; C07K 1/107; C12N 5/071

(52) U.S. Cl. 424/484; 530/350; 530/324; 435/397

(57) ABSTRACT

This invention relates to protein fibrils, to methods and kits of producing those protein fibrils comprising a plurality of first peptide monomer units arranged in a first strand and a plurality of second peptide monomer units arranged in a second strand in which said first and second strands form an overlapping staggered heterodimer coiled coil structure, and wherein the amino acid residues on the exposed surface of said first and second strands enable said protein fibril to interact with another protein fibril in a plurality of non-parallel orientations. This invention also relates to bundles of protein fibrils and matrices, in particular, hydrogels produced using those protein fibrils.

### FIELD OF THE INVENTION

This invention relates to protein structures, to methods of producing those protein structures, and to protein fibres and other materials, in particular, hydrogels produced using those protein structures.

### BACKGROUND

As our understanding of sequence-to-structure relationships in proteins improves, so does our ability to rationally design new proteins and protein-based materials. Unlike discrete peptide and protein objects, the design of biomaterials requires additional rules for self-assembly to allow the nano-to-micron scale regimes to be bridged (Woolfson & Ryadnov, 2006; Ulijn & Smith, 2008). In these respects, synthetically accessible peptides, which can be programmed to fold into prescribed structures and to self-assemble into larger architectures, offer routes to rationally designed peptide and protein-based biomaterials. Indeed, a variety of peptide-based self-assembling fibres, tapes and hydrogels have been produced (Zhang et al., 1993; Aggeli et al., 1997; Pandya et al., 2000; Hartgerink et al., 2001; Schneider et al., 2002; Paramonov et al., 2005). Much of this effort has been directed to the assembly of β-structured systems, though α-helix-based fibrous and α-helix-containing gelling materials have been explored to some extent (Pandya et al., 2000; Petka et al., 1998; Wang et al., 1999; Potekhin et al., 2001; Zimenkov et al., 2004; Dong et al., 2008; Gribbon et al., 2008).

In WO 2001/021646, the inventors described a self-assembling fibre (SAF) system enabling the sticky-end directed molecular assembly of α-helical coiled coils. The system comprises two short peptides (SAF-p1 and SAF-p2) of de novo design. The SAF-p1 and SAF-p2 sequences were designed to co-assemble, resulting in an offset α-helical dimer with complementary sticky ends. The ends promote longitudinal assembly into α-helical coiled-coil fibrils, which bundle to form matured fibres. Subsequently, the inventors introduced fibre-shaping peptides into the SAF system allowing morphological changes to be made to protein fibres comprising self-assembling peptides (WO 2004/022584). However, the previously described SAF system generally leads to highly ordered and thickened fibres which can settle out of solution (FIG. 1a). This is because the specific interactions fostered by features on the surface of the coiled coil tends to result in crystallization of the peptides within the fibres (Papapostolou et al., 2007).

Constructing novel three dimensional scaffolds to grow cells is crucial to properly understanding how they behave in living systems. There has been a lot of evidence that cells behave differently in 3 dimensions to 2 dimensions. Therefore, building 3-dimensional structures and eventually tissues that mimic the extracellular matrix to grow cells has been a goal for cell biologists for many years.

A single-peptide hydrogelating system, namely MAX3 and variants from the Schneider and Pochan team (Pochan et al., 2003) has been developed. In this system, the peptide is designed to form an antiparallel 13-hairpin, which folds unimolecularly upon heating and then assembles into amyloid-like fibrils that gel; the process reverses upon cooling.

However, there are a number of well-known diseases associated with 13-pleated sheets that are serious, including Alzheimer's, Parkinson's and Huntington's disease. Therefore, a hydrogelating system based on β-structures have the theoretical potential to cause unwanted diseases.

In contrast, the inventors in this case have developed a hydrogelating SAF system (hSAF) based on α-helical coiled coil structures.

### SUMMARY OF THE INVENTION

According to the first aspect of the invention there is provided a protein fibril comprising a plurality of first peptide monomer units arranged in a first strand and a plurality of second peptide monomer units arranged in a second strand in which said first and second strands form an overlapping staggered heterodimer coiled-coil structure, wherein the amino-acid residues on the exposed surface of said first and second strands enable said protein fibril to interact with another protein fibril in a plurality of non-parallel orientations.

The peptide monomer units according to the first aspect of the present invention are also referred to as hSAF peptides. A plurality of peptide monomer units means at least 2, preferably at least 7, more preferably at least 14, even more preferably at least 21, and particularly preferably at least 42 peptide monomer units. The protein fibril of the present invention is preferably at least 50 amino acids in length, more preferably at least 100 amino acids in length, yet more preferably at least 200 amino acids in length, still more preferably at least 500 amino acids in length, and particularly preferably at least 1000 amino acids in length.

The term “staggered heterodimer” refers to a dimeric structure formed by two different strands in which the two strands assemble to leave overlapping ends that are not interacting within the heterodimer. The staggered heterodimer is also referred to herein as a protein fibril.

The term “coiled coil” refers to a peptide/protein sequence usually with a contiguous pattern of hydrophobic residues spaced 3 and 4 residues apart, which assembles (folds) to form a multi-meric bundle of helices. Coiled coils including sequences with multiple offset repeats are also contemplated. Preferably, purely α-helical coiled-coils are formed in the protein fibril of the present invention.

The term “another protein fibril” refers to any other protein fibril that is capable of interacting with the protein fibril of the present invention. The “another protein fibril” may be a protein structure according to the original SAF design, i.e., one disclosed in WO 2001/021646 or WO 2004/022584 to result in hybrid materials consisting of a plurality of different types of protein fibrils and/or other materials. Preferably, the “another protein fibril” is a protein fibril according to the present invention. The protein fibrils according to the present invention interact with each other through interaction forces on the exposed surfaces of the protein fibrils. Said interaction forces can be any non-covalent intermolecular interaction forces, which include but are not limited to hydrophobic interactions, H-bond interactions and ionic interactions. Preferably, the interaction forces are hydrophobic and H-bond interactions. As it will be clear to one skilled in the art, the nature of the interaction force on the exposed surfaces of the protein fibrils is determined by the identity of the amino acid residues on the exposed surfaces of said first and second strands. For instance, for hydrophobic interactions, the residues may be any of the hydrophobic residues selected from alanine, valine, leucine, isoleucine, methionine, phenylalanine, tryptophan and tyrosine. The hydrophobic residues are preferably alanine, valine, leucine, isoleucine or tryptophan, or other structurally similar residues. More preferably, the hydrophobic residues are alanine or tryptophan. For H-bond interactions, the residues may be any of the uncharged polar residues selected from glutamine, asparagine, serine and threonine. The uncharged polar residues are preferably glutamine or asparagine. For ionic interactions, the charged residues may be selected from arginine and lysine (positively charged), or aspartic acid and glutamic acid (negatively charged).

The term “amino acid” embraces both naturally occurring amino acids, synthetic amino acids and naturally occurring amino acids that have been modified. In all cases references to naturally occurring amino acids may be considered to include synthetic amino acids which may be substituted therefor.

The exposed surface of the protein fibril of the present invention preferably comprises only one type of interaction force. For example, the exposed surfaces of both the first and second strands may have the same type of interaction force, e.g., either hydrophobic or H-bond interaction. However, the exposed surface of the protein fibril may comprise a plurality of different types of interaction forces. For example, the exposed surface of the first strand may have one type of interaction force, such as hydrophobic interaction, and the exposed surface of the second strand may have a different type of interaction force, such as H-bond interaction. Moreover, there may be a number of different interaction forces, e.g. both hydrophobic and H-bond interactions, on the exposed surface of the first and/or second strand.

The term “a plurality of non-parallel orientations” means that the protein fibrils interact in a plurality of orientations wherein the fibrils are not aligned in a parallel manner. In particular, the protein fibrils may interact so that only part of the protein fibrils interact. The non-interacting portions of the protein fibrils can form free ends or loop structures. Such interactions result in the formation of a matrix or network structure. As will be appreciated by those skilled in the art, there may be some level of parallel alignment between the protein fibrils, but the arrangements of the exposed amino acids encourage non-parallel interactions so that a matrix or network structure is formed by the protein fibrils.

The inventors have found that unlike the specific interactions fostered on the surfaces of the original SAF peptides (FIG. 1a), which results in parallel alignment between the protein structures and subsequent formation of thickened fibres, the more general interactions on the exposed surface of the hSAF peptides allow the protein fibrils of the present invention to interact with each other in a plurality of orientations, particularly in a non-parallel manner. This results in smaller, thinner and more flexible bundles of protein fibrils that are capable of forming matrices or networks of non-covalently cross-linked fibrils and, hence, physical hydrogels (Kope\check{c}ek & Yang, 2009; FIG. 1b).

A protein fibril according to the present invention may further comprise fibre-shaping peptides, i.e. a hub and a plurality of peptide monomer units each being attached at one end thereof to the hub, wherein the free ends of at least 2 peptide monomer units are N-termini or C-termini, and each of the at least 2 peptide monomer units is capable of interacting with said first or second strand. The presence of fibre-shaping peptides allows morphological changes to be made to the protein structures. In particular, the fibre-shaping peptides allow one to incorporate branches, splits, kinks and bends in the protein structures. Such fibre-shaping peptides are described in European Patent No. 1534741.

A protein fibril according to the present invention may be derivatised at some peptide monomer units. For example, fluorescent moieties (fluorophores) may be attached to the coiled coil as described in WO99/11774. The addition of fluorescent moieties may assist visualization of the protein structure. Other derivatives may include attaching metal particles, such as gold nanoparticles, and binders to the peptide monomer units for example so that units that can bind other entities can be produced. The peptide monomer units of the present invention may also be derivatised by macromolecules. Said macromolecules may be any functional macromolecules, which include but are not limited to extracellular matrix components, such as growth factors and polysaccharides, small peptides which bind to cell-surface proteins, such as cell adhesion motifs (e.g. RGD and IKVAV (Ile-Lys-Val-Ala-Val; SEQ ID NO:1) peptide), cytokines and hormones. The protein fibrils according to the present invention may be derivatised pre-, co- or post-assembly, or a combination thereof. Derivatisation pre-assembly involves derivatisation of the peptide monomer units before they are assembled into the protein fibrils. Derivatisation co-assembly involves derivatisation of the peptide monomer units whilst they are being assembled into the protein fibrils. Derivatisation post-assembly involves derivatisation of the protein fibrils after the peptide monomer units have been assembled into the protein fibrils. Preferably, derivatisation (functionalisation) occurs post-assembly as separating assembly and functionalisation allows the possibility of making general, and potentially more-versatile materials. Derivatisation of the peptide monomer units/protein fibrils with macromolecules allows the present invention to be suitable for use in various biotechnological and medical applications, such as cell growth, tissue engineering and drug delivery. It also provides better control over processes, such as cell growth. The choice of macromolecules by which the peptide monomer units are derivatised depends on the use of the present invention.

Methods for derivatising peptide monomer units or protein fibrils are well known to those skilled in the art. The peptide monomer units or protein fibrils may be derivatised through non-covalent or covalent binding. Derivatisation through non-covalent binding can be achieved, for example, using hydrophobic interactions, electrostatic interactions (such as negatively charged peptides (e.g. DE-based peptides), and neutral peptides (e.g. AQ-based peptides)), structural mimics and complementary pairing (Woolfson and Mahmound, 2010). Derivatisation through covalent binding can be achieved through, for example using recombinant expression, hybrid systems or click chemistry (Woolfson and Mahmound, 2010). In a preferred embodiment of the present invention, derivatisation is achieved using click chemistry, as described in Woolfson and Mahmound, 2010.

“Click chemistry” (also known as “click reactions”) is a term well-known to those skilled in the art. It refers to the concept of generating substances by joining small modular units together. Exemplary click reactions include, but are not limited to, Huisgen 1,3-dipolar cycloaddition (e.g. the copper(I)-catalyzed azide-alkyne cycloaddition), the Diels-Alder reaction, nucleophilic substitution (such as to small strained rings like epoxy and aziridine compounds), oxime ligation, hydrazone ligation, thiazolidine ligation, dihydroxylation (for addition to C═C) and thiol-yne reaction (for addition to alkynes). In particular, the peptide monomer units of the present invention may be derivatised so that they contain a first click group. The derivatised peptide monomer units can then be functionalised through conjugation with moieties/macromolecules having a complementary second click group using, for example copper-catalysed azide-alkyne or thiol-ene click reactions, to form functionalised peptide monomer units. Alternatively, the derivatised peptide monomer units containing the first click groups can co-assemble with non-derivatised (standard) peptide monomer units to produce protein fibrils of the present invention. The protein fibrils can subsequently be functionalised using click reactions as described above, thereby resulting in functionalised protein fibrils. Click chemistry combines high selectivity and high yields under ambient, biocompatible conditions. It allows high levels of incorporation of derivatised peptide monomer units (up to 100%), and subsequently high levels of coverage of protein fibrils with functional molecules, to be achieved. It also allows dual functionalisation of the protein fibrils (e.g. incorporating both gold nanoparticles and fluorophore labels into a single fibril). Derivatisation can be achieved using one type of click reaction or a combination of different types of click reactions (e.g. copper-catalysed azide-alkyne and thiol-ene click reactions). “Click groups” are groups that are suitable for any click reactions, such as those mentioned above. Suitable click groups are well known to those skilled in the art (see, for example, Shao et al., J. Am. Chem. Soc., 117, 3893-3899, 1995). For example, suitable click groups for Huisgen 1,3-dipolar cycloaddition may be azide and alkyne groups, those for the Diels-Alder reaction may be diene and dienophile groups, those for thiol-yne reaction may be thiol and alkyne groups, those for oxime ligation may be aldehyde and hydroxylamino groups, those for hydrazone ligation may be hydrazino and lipophilic glyoxylyl acid groups, those for thiazolidine ligation may be aldehyde and amino thiol groups, and those for thiol-ene reaction may be thiol and alkene groups. First and second click groups are complementary to each other such that click reactions can occur between the groups. For example, in a Huisgen 1,3-dipolar cycloaddition reaction, the first click group may be an azide group, and the second click group may be an alkyne group. It will be appreciated by those skilled in the art that the first and second click groups may be reversed. The click groups can be spaced from the peptide monomer units and/or protein fibrils using spacers well known to those skilled in the art.

The peptide monomer units of the present invention may comprise a repeating structural unit. Preferably, the repeating structural unit comprises a heptad repeat motif (abcdefg). Other repeats (e.g hendecads—abcdefghijk) and amino acid compositions may also be used (see WO99/11774).

In a heptad repeat, the a, d, e and g positions are responsible for directing the dimer interface and the amino acid identities of these positions are the same as the original SAF design (see WO2001/021646 and WO2004/022584). In brief, there are mostly hydrophobic residues at the a and d positions of the first and second monomer units and charged amino acid residues at positions e and g in each first peptide monomer unit and oppositely-charged amino acid residues at positions e and g in each corresponding second peptide monomer unit, to ensure that the first strand and the second form a staggered parallel heterodimer coiled coil structure. The hydrophobic residues are preferably valine, leucine, isoleucine or other structurally similar amino acid residues. The charged residues are preferably selected from arginine and lysine residues (positively charged), or glutamic acid and aspartic acid residues (negatively charged). A pair of asparagines (or other suitable polar residues) are provided at complementary a positions in complementary heptad repeats within corresponding first and second monomer units to assist with forming the parallel heterodimer coiled coil structure.

In the heptad repeat, positions b, c and f are exposed on the surface of said first and second strands when the heterodimer coiled-coil structure is formed. It is therefore preferred that the heptad repeat comprises, at the b, c and f positions, hydrophobic residues for hydrophobic interactions, uncharged polar residues for H-bond interactions, or charged residues for ionic interactions.

The residues at the b, c and f positions may be the same or different so long as they allow the protein fibril to interact with another protein fibril in a plurality of non-parallel orientations as mentioned above. In a preferred embodiment of the present invention, the residues at the b, c and f positions in a single repeat are characterised by the same type of side chains. For instance, they all have hydrophobic side chains, uncharged polar side chains, positively charged side chains or negatively charged side chains.

Moreover, the residues at any of the b, c and f positions of one protein fibril are capable of interacting with the residues at any of the b, c or f positions of another protein fibril. Preferably, the residue at the f position of one protein fibril interacts with the residue at the b or c position of another protein fibril in order for the protein fibrils to interact in a non-parallel manner.

In the original SAF design, the protein structures do not exhibit a general interaction force across the exposed surfaces. Instead the exposed amino acids encourage specific interactions at the b and c positions and forces the protein structures into a substantially parallel association/interaction, thereby leading to peptide alignment and fibre thickening. In contrast, the present invention provides protein fibrils in which the amino acid residues at the b, c and f positions of at least some of the peptide monomers have the same type of side chains, hence enabling a general interaction force across the exposed surface of those parts of the protein fibrils. Such a general interaction force encourages association of protein fibrils in a non-parallel manner, resulting in smaller and more flexible bundles of protein fibrils, which can then associate to form a matrix structure.

In one embodiment, the amino acid residues at the b, c and f positions are chosen to promote weak non-covalent interactions between protein structures. The term “weak non-covalent interaction” means that the interaction is relatively unstable. Residues with small hydrophobic side chains, such as alanine or other residues with similar structures, may be chosen to promote weak hydrophobic interactions for this purpose. In a preferred embodiment, alanine can be used at all three positions (i.e., b, c and f). Protein fibrils with such weak surface interactions enable the reversible formation of matrix (e.g. hydrogels) in a controlled manner.

In another embodiment, the amino acid residues at the b, c and f positions are chosen to promote stronger non-covalent interactions so as to result in stabilised gels. Such interactions may be multiple hydrophobic interactions, H-bond interactions or ionic interactions. In case of multiple hydrophobic interactions, at least one position on the exposed surface may be occupied by a more-hydrophobic residue, i.e. a residue with a large hydrophobic side chain, such as tryptophan, phenylalanine or tyrosine, preferably tryptophan. Said at least one position can be any of the b, c or f positions, preferably it is the f position. For H-bond interactions, glutamine is preferably used at least one of the positions, more preferably at least two of the positions and most preferably at all three positions (i.e., b, c and f).

In a preferred embodiment of the present invention, the protein fibril is formed entirely of heptad repeats in which every single heptad repeat has either alanine at all b, c and f positions or glutamine at all b, c and f positions. In another preferred embodiment of the present invention, the protein fibril is formed entirely of heptad repeats in which every single heptad repeat has alanine at the b and c positions, and tryptophan at the f position.

In the protein fibrils according to the above preferred embodiments, the first and second peptide monomer units may have the following sequences:

(hSAF_{AAA} p1; SEQ ID NO: 2)
a) KIAALKAKIAALKAEIAALEAENAALEA
and
(hSAF_{AAA} p2; SEQ ID NO: 3)
b) KIAALKAKNAALKAEIAALEAEIAALEA
respectively; or
(hSAF_{AAA-W} p1; SEQ ID NO: 4)
c) KIAALKAKIAALKAEIAALEWENAALEA
and
(hSAF_{AAA-W} p2; SEQ ID NO: 5)
d) KIAALKAKNAALKAEIAALEWEIAALEA
respectively; or
(hSAF_{QQQ} p1; SEQ ID NO: 6)
e) KIQQLKQKIQQLKQEIQQLEQENQQLEQ
and
(hSAF_{QQQ} p2; SEQ ID NO: 7)
f) KIQQLKQKNQQLKQEIQQLEQEIQQLEQ
respectively.

It will be appreciated that these are examples only of 4-heptad structures and that other lengths are possible and envisaged for use in the invention.

One or more amino acid residues (e.g., one, two or three amino acid residues) may be substituted with an amino acid that has been derivatised (e.g., derivatised with a click group). Substitution may occur at any position and by any derivatised amino acid provided that the peptides are still able to interact to form protein fibrils. Preferably, the amino acid residue alanine or glutamine is substituted. More preferably, alanine is substituted. The amino acid residues are preferably substituted by a derivatised lysine residue. In addition, substitution can occur at any position in the peptide. Preferably, substitution occurs near or at the C-terminus of the heptad (i.e., the f position). More preferably, substitution occurs near or at the C-terminus of the most C-terminal heptad (i.e. the fourth heptad). For example, the C-terminal alanine residue in peptide c) may be substituted by a derivatised lysine residue, resulting in a peptide having the amino acid sequence KIAALKAKIAALKAEIAALEWENAALEK (SEQ ID NO: 8). For example, the C-terminal lysine residue (which substitutes an alanine residue in the c) peptide) may be derivatised by a click group, such as an azide group (resulting in peptide hSAF-p1N₃ having the amino acid sequence KIAALKAKIAALKAEIAALEWENAALEKz (SEQ ID NO: 9), wherein Kz refers to a lysine residue having an azide side chain). The inventors have found that the azido-lysine residue at the C-terminus of the peptide does not disrupt the regular coiled-coil heptad repeat of the peptide.

In a hendecad repeat, the a, b, d, e, h and i positions are responsible for the formation of heterodimers; and the c, d, g, k and j positions of the peptide monomers are on the exposed surface of the protein fibril. Similar amino acid residues as mentioned for the b, c and f positions in a heptad repeat can be chosen for the c, d, g, k and j positions in a hendecad repeat according to the present invention.

The stability of the individual protein fibrils may be improved by making the peptide monomers longer, such that the overlap between corresponding first and second monomer unit residues is increased. Increases in monomer length have previously been shown to stabilize coiled coil structures. Alternatively, stability can be improved by introducing bonding between adjacent peptide monomer units in the same strand. For example, Dawson and co-workers (1994) have produced peptide bonds between adjacent polypeptide units by coupling and subsequent rearrangement of a cysteine residue at the N end of one polypeptide unit to a thio-ester derivatised C-terminus of another unit.

According to another aspect of the invention, there is provided a method of producing protein fibrils of the present invention, the method comprising providing the first and second peptide monomer units that associate to form a protein fibril according to the invention and mixing said first and second peptide monomer units together.

The first and second peptide monomers and the strands may have the characteristics described above. In particular, the first and second peptide monomer units may be derivatised as mentioned above. Derivatisation may be performed before or after assembly of the peptide monomer units into the protein fibril of the invention.

According to yet another aspect of the invention, there is provided a kit for making a protein fibril, the kit comprising first and second peptide monomer units which, upon mixing, associate to form a protein fibril according to the invention.

According to still another aspect of the invention there is provided a bundle of protein fibrils produced by an association of a plurality of protein fibrils according to the invention, wherein at least one protein fibril of said bundle associates with another protein fibril of said bundle in a plurality of non-parallel orientations.

Thinner and more flexible bundles of protein fibrils result from the present invention, in comparison with the highly ordered and thickened protein fibres, which tend to settle out of solution, produced by the original SAF system. This is because, in the original SAF design, the specific charged interactions between certain b and c positions lead to peptide alignment and fibre thickening; whereas in the protein fibrils of the present invention, said specific interactions are replaced with more general interactions on the exposed surface thereof. This leads to networks of non-covalently cross-linked fibrils.

According to a further aspect of the invention there is provided a three dimensional matrix produced by an association of a plurality of protein fibrils or a plurality of bundles of protein fibrils according to the present invention. In one embodiment of the present invention, the three dimensional matrix produced is a biocompatible hydrogel when the matrix is formed in the presence of an aqueous solution under conditions which promote the formation of hydrogels. Such conditions are well-known to those skilled in the art.

The term “hydrogel” refers to a gel structure with a high water content. The water content in a hydrogel is at least 85%, preferably at least 90%, more preferably at least 95%, still more preferably at least 99%. The resulting hydrogel has a wide variety of potential applications in biotechnology and medicine, such as the controlled delivery and release of cells, cosmetics and drugs; and as supports for cell growth and tissue engineering.

The inventors have found that the nature of the interactions on the surfaces of the protein fibrils determines the underlying mechanism of gelation, and consequently, the properties of the hydrogels formed from the protein fibrils. For instance, hSAF peptides that have surface polar residues with H-bonding potentials assemble to weak gels at low temperatures, for example, at or below 12° C., preferably below 10° C., more preferably below 5° C. and yet more preferably at 0° C. The resulting hydrogels melt on warming up to 35° C. and reform at temperatures above 35° C. On the other hand, hSAF peptides that have hydrophobic residues on the outer surfaces form gels at low temperatures and remain as gels at all temperatures. The resulting hydrogels become stronger on warming and are stable up to at least 95° C. Therefore, hydrogels with specific properties can be engineered using peptide monomers with particular amino acid residues on the exposed surfaces of the protein structures according to the present invention.

In addition, the stability of individual bundles of protein fibrils and of the matrix/hydrogel structure formed by the protein fibrils of the present invention may be improved by covalent cross-linking between the protein fibrils or bundles of protein fibrils.

In a preferred embodiment, a hydrogel is assembled in situ. For example, a hydrogel can be formed in a solution to entrap contaminants in the solution and then the hydrogel, together with contaminants, can be removed from the solution for example by centrifugation.

The hydrogel according to the present invention may further comprise macromolecules. Said macromolecules may be covalently linked to the peptide monomer units through derivatisation as mentioned above. Alternatively, said macromolecules may not be covalently linked with the protein fibrils, but instead they may be trapped in the aqueous portion of the hydrogels.

The inventors have found that decorated proteinogenic scaffold (i.e. the functionalised hydrogel) enhances matrix-cell interaction and influences cellular behaviour.

The present invention also provides a method of producing a bundle of protein fibrils, the method comprising:
a) providing the first and second peptide monomer units which associate to form a protein fibril and/or a bundle of protein fibrils according to the invention and mixing said first and second peptide monomer units together; or
b) providing a mixture of protein fibrils according to the invention,
   and the accumulation and/or assembling of such protein fibrils forming a bundle of protein fibrils.

The present invention further provides a method of producing a three dimensional matrix, the method comprising:
a) providing the first and second peptide monomer units which associate to form a protein fibril according to the invention and mixing said first and second peptide monomer units together; or
b) providing a mixture of protein fibrils according to the invention,
   and the accumulation and assembling of such protein fibrils or bundles of protein fibrils forming a three dimensional matrix.

An advantage of the method for making a three dimensional matrix, in particular a hydrogel, according to the present invention is that such a matrix/hydrogel is only formed upon mixing of the first and second peptide monomer units. This allows the controlled assembly of the matrix/hydrogel.

The method may further comprise a step of adding macromolecules (described above) to the mixture.

According to another aspect of the invention there is provided a kit for making a three dimensional matrix, the kit comprising:
a) first and second peptide monomer units which, upon mixing, associate to form a protein fibril or a matrix according to the invention; or
b) a mixture of protein fibrils according to the invention.

The kit may further comprise macromolecules as mentioned above.

According to another aspect of the present invention, there is provided a self-assembling peptide monomer unit derivatised with a click group.

According to another aspect of the present invention, there is provided a protein fibril comprising a plurality of first self-assembling peptide monomer units arranged in a first strand and a plurality of second self-assembling peptide monomer units arranged in a second strand in which said first and second strands form an overlapping staggered heterodimer coiled-coil structure, wherein at least one self-assembling peptide monomer unit is derivatised with a click group.

Derivatisation may occur in any of the first self-assembling peptide monomer units and/or any of the second self assembling peptide monomer units, as described above.

The term “self-assembling peptide monomer unit” is as defined in WO 2001/021646 and WO 2004/022584, and also includes the hSAF peptides according to the present invention. Preferably, the self-assembling peptide monomer units are the hSAF peptides according to the present invention.

Click groups are as described above.

According to yet another aspect of the present invention, there is provided a method of derivatising a self-assembling peptide monomer unit using click chemistry comprising incorporating a first click group into the peptide monomer unit, reacting the resultant peptide monomer unit with a molecule having a second click group which is complementary to the first click group, such that the monomer unit is attached to the molecule through the first and second click groups.

The resultant derivatised self-assembling peptide monomer unit may co-assemble with other derivatised or non-derivatised self-assembling peptide monomer units to form self-assembling fibres.

According to another aspect of the present invention, there is provided a method of derivatising a protein fibril using click chemistry comprising incorporating a first click group into at least a first and/or at least a second self-assembling peptide monomer unit, mixing the at least first and/or the at least second self-assembling peptide monomer unit having the first click group with a plurality of first and second self-assembling peptide monomer units together, reacting the resultant protein fibril with a molecule having a second click group which is complementary to the first click group, such that the protein fibril is attached to the molecule through the first and second click groups.

The first and second click groups may be groups suitable for any click reactions as described above, provided that the first and second click groups are complementary such that click reactions can occur between the groups. In some embodiments the first and second click groups are azide-alkyne and/or thiol-ene units. Preferably, they are azide-alkyne units.

A molecule having a second click group may be any molecule described above. It includes, but is not limited to, fluorophores, metal particles, binders and macromolecules.

### BRIEF DESCRIPTION OF THE FIGURES

FIG. 1A-1B illustrates the hSAF design principles. (FIG. 1A) In previous SAF designs, specific charged interactions between certain b and c positions lead to peptide alignment and fibre thickening. (FIG. 1B) For the hSAFs, the inventors replaced these specific interactions with weaker, more-general interactions at all b, c and f sites, to result in smaller, more flexible, bundles of thinner fibres.

FIG. 2A-2B demonstrates the self-supporting hydrogel formation by hSAFs. Vials were (FIG. 2A) incubated for 30 minutes on ice, inverted and then incubated on ice for a further 30 minutes, or (FIG. 2B) incubated for 5 minutes on ice, 25 minutes at 20° C., inverted and then incubated for a further thirty minutes at 20° C. All samples were 1 mM in each peptide.

FIG. 3 shows changes in elastic and viscous moduli with temperature using particle-tracking microrheology (PTM).

FIG. 4A-4E shows gel strength and network formation by the hSAFs. (FIG. 4A) Gel strength gauged by non-destructive oscillatory rheology. Key: G′ (solid symbols) and G″ (open); hSAF_{AAA} (discs) and hSAF_{QQQ} (squares). Measurements were made at a strain of 0.5%. (FIG. 4B-FIG. 4E) Network formation as observed by cryoSEM for: hSAF_{QQQ} (FIG. 4B), hSAF_{AAA} (FIG. 4C), and hSAF_{AAQ} (FIG. 4D), all assembled on ice for 15 minutes; and (FIG. 4E) for hSAF_{AAA} assembled on ice for 3 minutes and then at room temperature for 12 minutes. All images are at the same magnification.

FIG. 5 shows the TEM micrograph of 200 mM hSAF_{AAQ} fibres stained with uranyl acetate.

FIG. 6A-6D shows α-helical secondary structure and packing within the fibrils and gels. CD spectra (FIG. 6A and FIG. 6C) and x-ray fibre diffraction patterns (6B and 6D) for hSAF_{AAA} (FIG. 6A and FIG. 6B) and hSAF_{QQQ} (FIG. 6C and FIG. 6D). In 6B and 6D, M and E refer to meridional and equatorial reflections, respectively.

FIG. 7A-7B shows α-Helical secondary structure and packing in hSAF_{AAQ}. CD spectra (FIG. 7A) and x-ray fibre diffraction pattern (FIG. 7B).

FIG. 8A-8B demonstrates the characterisation of hSAF_{AAA-W}. (FIG. 8A) CD spectra showing α-helical secondary structure. (FIG. 8B) TEM of 750 μM hSAF_{AAA-W} fibres stained with uranyl acetate (scale bar: 500 nm).

FIG. 9 demonstrates the Alamar Blue proliferation assay of PC12 cells on hSAF_{AAA-W} gels. PC12 cells were exposed to different concentrations of hSAF_{AAA-W} peptides (from 0.5 mM to 2.5 mM). Results are expressed as percentage of fluorescence from cells incubated with hSAF_{AAA-W} versus no-peptide controls (100%). Mean+/−s.d. mean for n=3 experiments. There was no statistical difference among the controls and the test cells after 72 hours (one-way ANOVA test, Fisher's F-value<0.27, p=0.92). Staurosporine was used to establish definite cell death.

FIG. 10A-10D shows cell growth and differentiation on hSAF hydrogels. Phase-contrast microscopy of differentiating rat adrenal pheochromocytoma (PC12) cells in hSAF_{AAA-W} hydrogel (FIG. 10A), and Matrigel (FIG. 10B). These images were taken 10 days after adding nerve growth factor. Images from the full 14-day time course are given in the Supplementary information. Cell differentiation as observed by neurite outgrowth was semi-quantified over time by: (FIG. 10C) the lengths of the processes; and (10D) the percentage of cells showing processes. In both plots, error bars show the standard error of the mean for measurements from ≧100 cells/cell clusters across 10 different fields of view in three different triplicate wells.

FIG. 11 shows phase-contrast micrographs demonstrating the time course of differentiating PC12 cells on hSAF hydrogels and Matrigel. NGF was added on day 0. Note that even without adding NGF (day 0), some of the cells grown on Matrigel were differentiated as observed by early neurite formation. Cells shown on hSAF hydrogel started to differentiate on day 4-5, and this neared completion by at day 10. 3D clustering of cells were noted in both media.

FIG. 12A-12B confirms the covalent attachment of the biomimetic peptide to the gel. A chunk of functionalised gel was disassembled using 20% acetonitrile and 0.1% TFA and analysed using high-pressure liquid chromatography (HPLC) and matrix-assisted laser desorption ionisation—time of flight (MALDI-TOF) mass spectroscopy. FIG. 12A and FIG. 12B. Analytical HPLC tracing of hSAF-azide-RGD and hSAF-azide-IKVAV gels, respectively. p1KN₃ and p₂ represent the peptide with attached azide and the complementary peptide, respectively while p1KN₃—RGDS and p1KN₃—IKVAV are the corresponding click products after addition of the biomimetic peptide.

FIG. 13A-13B shows the extent of functionalisation in the gel. RGDS addition on the assembled fibre did not happen only on the surface of the gel but even to the bottom part as shown in this experiment. FIG. 13A. hSAF gel was prepared in a Pasteur pipette and was functionalised with RGDS peptide via click chemistry. (The arrow points to the upper surface of the gel.) FIG. 13B. The gel was then sectioned and each chunk was disassembled using 20% acetonitrile and 0.1% TFA and subsequently analysed using HPLC and MALDI-TOF mass spectroscopy. FIG. 13C and FIG. 13D. HPLC traces of uppermost and lowermost gel chunks, respectively.

FIG. 14A-14G shows cellular response of functionalised gels. FIG. 14A. One side of the well was filled with hSAF-azide gel and functionalised with RGDS biomimetic. The other half was non-functionalised and served as control. The gels were seeded with rat pheochromocytoma PC12 cells and induced to differentiate by adding NGF (100 ng/ml). FIG. 14B to FIG. 14D. Phase contrast images of PC12 cells on day 9. PC12 cells grown on hSAF-RGDS showed extensive neurite formation (FIG. 14B) compared to those seeded on plain scaffolds (FIG. 14D). The cells which landed between the two gels showed preferential neurite growth towards the functionalised gel (FIG. 14C and FIG. 14F). (The white arrow points to the boundary.) FIG. 14E to FIG. 14G. Similar experiment was done on rat primary hippocampal cells and maintained on serum-free media. Longer neurites were noted on hippocampal cells grown on functionalised gel (FIG. 14E) compared to plain scaffolds (FIG. 14G).

FIG. 15 shows that human fibroblasts seeded on decorated hSAF gel (right) have more stretched morphology compared to those grown on plain scaffolds (left).

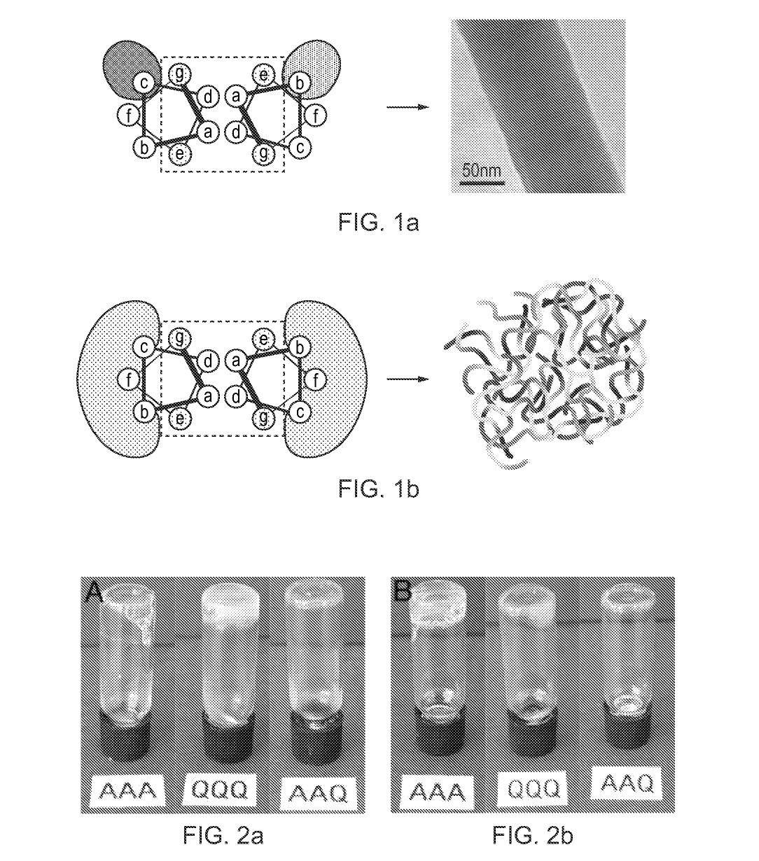

### MATERIALS AND METHODS

### Peptide Synthesis

Peptides were synthesized on a CEM “Liberty” peptide synthesizers using standard solid-phase Fmoc chemistry. Amino acids were purchased from Novabiochem and other reagents from Rathburn Chemicals unless otherwise stated. For synthesis of C-terminal modified peptide hSAF-p1N₃, the Fmoc-amino acids, Fmoc-Lysine-ε-azide were manually loaded onto Tentagel R PHB resin using 3 equivalents amino acid, 2.9 equivalents HBTU, 3 equivalents HOBt, 4.5 equivalents DIPEA and 0.1 equivalent DMAP. The resin was then washed with excess DMF, and loaded onto the synthesizer for further automated synthesis. Peptides were cleaved using 95% TFA (Sigma), 2.5% TIS (Sigma) and 2.5% 18.2 MΩ ultra-pure water, purified by reverse-phase HPLC using acetonitrile (Fisher)-water gradients with 0.1% TFA, and confirmed by MALDI-TOF mass spectrometry. Pure peptides were freeze dried from acetic acid, weighed and dissolved in ultra-pure water to give 3 mM (˜9 mg/ml) stocks.

Synthesis of Lysine ε-azide

Fmoc-Lysine ε-azide was prepared using the method of Goddard-Borger and Stick, 2007. Fmoc-Lys-OH.HCl (4.2 mmol, 1.79 g) was dissolved in methanol (100 ml). Potassium carbonate (2.03 g) and copper sulfate (20 mg) were added. Imidazole-1-sulfonyl azide hydrochloride (1.06 g) was added and the mixture stirred at RT for 12 hours. The solvent was removed in vacuo, and the residue partitioned between water (200 ml) and chloroform (150 ml) containing isopropanol (50 ml). The organic layer was separated and dried over MgSO4, and the solvent removed in vacuo. The crude product was purified using a short plug silica column. The crude material was loaded and washed with a 5% solution of acetone in dichloromethane (500 ml) and eluted with a 20% solution of acetone in dichloromethane (500 ml) to yield the desired compound in 55% yield.

### Preparation of Alkyne Derivatives

Alkyne functionalised peptides are prepared by coupling propiolic acid to the N-terminus of the resin attached biomimetic peptide (0.1 mmol). Conditions used were: 5 eq propiolic acid, 4.9 eq ethyl-2-ethoxy-1,2-dihydro-1-quinoline-carboxylate (Merck)(EEDQ), and 5 eq N,N-diisopropyl ethylamine in DMF (5 mL).

### Hydrogel Preparation

Hydrogels are assembled under sterile conditions. Peptides p1-azide and p2 are prepared in ice-cold 10 mM MOPS (Sigma) pH 7.4 and mixed on ice, with pipetting, at a resulting concentration of 1 mM (˜3 mg/ml) each peptide. The solution is gently delivered on one side of the well. This is allowed to stay on ice for 5 minutes and transferred to room temperature for 25 minutes. The gel is incubated overnight at 37° C.

### CD Spectroscopy

hSAF gels were prepared in 10 mM MOPS (Sigma) pH 7.0, at 1 mM (˜3 mg/ml) of each peptide and incubated as described. hSAF solutions (containing fibers) were prepared in the same conditions at lower concentrations (i.e., 100 μM) of each peptide compared to hSAF gel. CD experiments of gel and solution were carried out in 0.01 cm quartz cells (Hellma) and 0.1 cm quartz cells (Starna) respectively, using a Jasco J-810 circular dichroism spectrometer fitted with a Peltier temperature controller. Spectra were recorded between 190 and 260 nm with a 1 nm data pitch and bandwidth, 4 s response time, 50 nm.min⁻¹ scanning speed and averaged over 2 accumulations. Baselines recorded using the same buffer, cell and parameters were subtracted from the data.

### Post-Assembly Decoration of Fibers and Gels

Peptide 1 and peptide 2 were assembled as above. 1 mM CuSO₄ was pre-mixed with 1 mM alkyne-peptide. 1 mM ascorbic acid was mixed with this solution, which was subsequently added to fibres or gels. To achieve best coverage of decoration, the reaction was allowed to proceed at room temperature for 3 h in case of fibres and for 24 h in case of gels. The fibres are subsequently spun to remove excess Cu, followed by several washes with PBS. The gel is subsequently washed at least 10 times with 10 mM EDTA (Sigma), followed by 10 washes in PBS.

### Electron Microscopy

Low Temperature Field Emission Scanning Electron Microscopy was carried out using a JEOL 6301F microscope and Gatan Alto 2500 low temperature equipment. Samples were mounted on brass rivets of 1 mm internal diameter. At set times, samples were frozen in nitrogen slush, and stored at −80° C. These were mounted into a cooled holder (−196° C.), plunged into liquid nitrogen, and transferred to the preparation chamber. These were then fractured using a cold scalpel tip and warmed to −90° C. for 30 seconds to remove a layer of ice. After re-cooling to −110° C., samples were coated with 2 nm Pt/Pd and transferred to the microscope stage. For TEM experiments, 6 μl samples were placed on carbon-coated 400-mesh copper grids (Agar) on filter paper and dried; stained with 6 μl of filtered 1% uranyl acetate solution; and examined in a Philips CM 100 microscope at 80 kV. Images were recorded using a Kodak Megaplus Camera 1.4i digital camera.

### Rheology

Rheological measurements used an Anton Paar Physica RC 301 with 8 mm parallel plate geometry, Peltier plate, and environmental hood, with a 200 mm gap setting. G′ and G″ were recorded using non-destructive oscillatory measurements at a strain of 0.5%, and over 3-45° C. The measuring plate was surrounded by water to prevent drying of the sample. Samples were mixed in situ on the lower plate at 3° C. to total volumes of 300 μl, the geometry was lowered into position and samples incubated for 30 minutes.

### Microrheology

Gel strength was probed using particle-tracking microrheology (PTM), in order to detect any difference between the behaviour of the material at the bulk and micro scales. PTM is a non-invasive technique for measuring local viscoelasticity in complex fluids (Cicuta & Donald, 2007). PTM directly observes the motion of embedded probe particles via optical microscopy. The mean square displacement (MSD) of the particles is calculated and related to the viscoelastic modulus of the sample using a Generalised Stokes-Einstein Relation (Mason, 2000; Hasnain & Donald, 2006). PTM is useful for studying the sol-gel transition of viscoelastic materials (Larsen & Furst, 2008) particularly for weak or incipient gels that may be disrupted by bulk shear rheometry. The spatial information for individual probe particles can be used to quantify the degree of heterogeneity in the system (Houghton et al., 2008).

Experiments were carried out using a Zeiss LSM 510 confocal microscope operated in bright-field mode with a 50× objective. The temperature was controlled using a Linkam heat stage with a liquid nitrogen attachment for cooling. The probe particles were 1 μm diameter amine-modified latex spheres (Sigma) added at a volume fraction of 10⁻⁴. Videos were captured in uncompressed avi format at 640×480 resolution and analysed using custom-written Matlab scripts. Viscoelastic moduli were calculated by taking Laplace Transform of the MSD and using GSER. Elastic and loss moduli extracted using analytic continuation into the complex plane (Hasnain & Donald, 2006 (see above)). The frequency of interest was chosen as 5 Hz because the more conventional 1 Hz was affected by truncation errors when taking the Laplace Transform due to the finite length of the data (Mason et al., 1997). The tracking resolution obtainable depends on lighting conditions (Papagiannopoulos, 2008; Savin & Doyle, 2005), and is typically 1/30 pixel. This resolution was reduced to ˜ 1/10 pixel by sample opacity and condensation at low temperatures, reducing the maximum measurable modulus to around 10 Pa. Close to resolution limit, values of the elastic modulus (G′) should be interpreted as lower limits.

### XRD

Samples for X-ray fibre diffraction were prepared as previously described (Papapostolou et al., 2007). 10 μl of the hydrogels were suspended between two wax-filled capillaries, spaced ˜1 mm apart and allowed to dry in air. The capillaries were gently separated and the fibre samples placed on a goniometer head. Diffraction data were collected using an R-Axis IV++ detector and Rigaku rotating anode with CuKa radiation. The specimen-detector distance was 160 mm, and exposure time 15 mins. X-ray patterns were examined using Mosflm (Winn, 2003) and CLEARER (Makin et al., 2007).

### Cell Biology

Rat adrenal pheochromocytoma (PC12) cells and the subclone Neuroscreen-1 (NS-1) cells (ThermoFisher Scientific Cellomics) were used. Cells were maintained at 37° C., 5% CO₂ in cell-culture medium comprising: Dulbecco's Modified Eagle Media (DMEM, Invitrogen) supplemented with 10% (v/v) horse serum (Sigma), 5% (v/v), fetal bovine serum (Sigma), 1% (v/v) penicillin/streptomycin solution (Sigma), and 2 mM L-glutamine (Invitrogen). For the proliferation assays, cells were seeded at 1×10⁴ cells cm⁻² in 96-well plates pre-coated with collagen solution (4 mg rat-tail type VII collagen (Sigma) dissolved in filter-sterilized solution of 0.8 ml glacial acetic acid, 100 mls ultra-pure H₂O; and activated before coating with 40 μl sterile 3.7% (w/v) NaCl per ml of collagen solution). After incubation for 24 hours, the medium was replaced, and peptide (0.5 mM, 1.0 mM, 1.5 mM, 2.0 mM. 2.5 mM total) added. After 72 hours, Alamar Blue dye (Serotec) was added (10% (v/v)) and incubated for 8 hrs. The fluorescence (excitation, 560 nm; emission, 590 nm) of 100 μl samples was measured. The ratio of the fluorescence intensities to that for a no-peptide control wells was taken as a measure of cell proliferation. Another control used staurosporine (Sigma) to establish cell death. To induce differentiation, nerve growth factor (Sigma) is added at 100 ng/ml in DMEM. Media are replaced every 3-4 days. Hippocampal cells were dissected from the brains of E18 Wistar rat embryos. The isolated tissue was then enzymatically dissociated using trypsin treatment (0.5 mgml⁻¹ for 9 min) followed by mechanical dissociation. Cells were then plated on poly-L-lysine-coated glass coverslips at a density of ˜100 cells per mm² and grown under standard cell culture conditions (5% CO₂, 37° C.). The culture medium was composed of Neurobasal medium (Gibco Invitrogen) supplemented with horse serum (9%), B27 (Gibco Invitrogen) and 2 mM glutamine. On the second day, the media was changed to Neurobasal medium supplemented with B27 and 2 mM glutamine and neurons were then fed each week with this serum-free medium. To introduce GFP, 2 μl of Sindbis virus expressing free GFP was added to cultures 8-12 hours before imaging. Human fibroblast cells were given as gift by Dr Mistry (Dept of Cellular and Molecular Medicine, University of Bristol). They were maintained under standard cell culture conditions with Fibroblast Basal Medium with supplements and penicillin/streptomycin) (Lonza). All cells were seeded on the gels at 10,000 cells/ml concentration. Cell morphology was followed up using Leica Microsystem Wetzlar GmbH (090-135.001) and images recorded using Canon PowerShot S70 7.1 megapixels.

Matrigel (BD Biosciences) was prepared following manufacturer's instructions, before washing and seeding similarly. Cells were followed for 2 weeks, replacing with fresh medium and NGF every 3-4 days.

### DETAILED DESCRIPTION OF THE INVENTION

Initially, three hSAF designs were investigated: variants hSAF_{AAA}, hSAF_{QQQ} and hSAF_{AAQ}, as shown in Table 1, where subscripts denote amino acids at b, c and f, respectively. In hSAF_{AAQ}, which serve as a control, the pattern of alanine and glutamine residues was the same as for canonical positions in previous SAF designs.

TABLE 1

| | Sequence | SEQ |
|---|---|---|
| Peptide name | g abcdefg abcdefg | ID |
| Heptad repeat | abcdefg abcdef | NO: |
| hSAF_{AAA} p1 | K IAALKAK IAALKAE | 2 |
| | IAALEAE NAALEA | |
| hSAF_{AAA} p2 | K IAALKAK NAALKAE | 3 |
| | IAALEAE IAALEA | |
| hSAF_{QQQ} p1 | K IQQLKQK IQQLKQE | 6 |
| | IQQLEQE NQQLEQ | |
| hSAF_{QQQ} p2 | K IQQLKQK NQQLKQE | 7 |
| | IQQLEQE IQQLEQ | |
| hSAF_{AAQ} p1 | K IAALKQK IAALKQE | 10 |
| | IAALEQE NAALEQ | |
| hSAF_{AAQ} p2 | K IAALKQK NAALKQE | 11 |
| | IAALEQE IAALEQ | |
| hSAF_{AAA-W} p1 | K IAALKAK IAALKAE | 4 |
| | IAALEWE NAALEA | |
| hSAF_{AAA-W} p2 | K IAALKAK NAALKAE | 5 |
| | IAALEWE IAALEA | |

Complementary hSAF peptides were mixed on ice, and either allowed to assemble at this temperature for 30 minutes, or removed after 5 minutes and incubated for 25 minutes at 20° C. After these times, to test for gel formation, sample vials were inverted and incubated for a further 30 minutes without changing the temperature (FIG. 2). Through this simple test, hSAF_{AAA} and hSAF_{QQQ} both formed self-supporting gels. hSAF_{QQQ} formed a gel at low temperature, which melted on warming; whereas, the hSAF_{AAA} appeared to form a weak gel at low temperature that strengthened on warming. Moreover, this gel did not melt on heating up to 95° C. The control, hSAF_{AAQ} did not form gels.

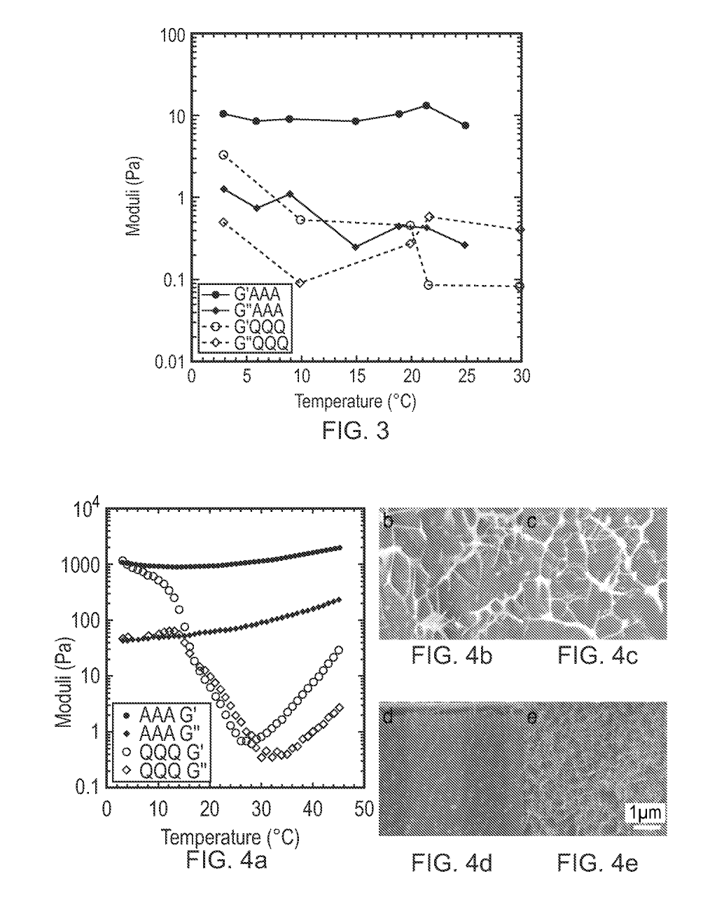

To confirm and quantify the gel strengths, the storage (G′) and loss (G″) moduli were recorded as a function of temperature using both microrheology (FIG. 3) and bulk oscillatory rheology, FIG. 4a. The results from PTM compared well to those for the bulk rheology, hSAF_{QQQ} showed a loss of structure between 13° C. and 20° C. while hSAF_{AAA} remained a gel at all temperatures (FIG. 3). The value of G′ for hSAF_{AAA} was higher than the resolution of the PTM technique, and should be viewed as a minimum value rather than an absolute. Above 35° C., hSAF_{QQQ} reformed into a heterogeneous gel, however, the heterogeneity and increased opacity of the sample made calculation of values for G′ and G″ unreliable. As with the bulk rheology, hSAF_{AAQ} and the majority of the individual peptides did not form gels, but behaved as viscous fluids. The exception was hSAF_{AAA}-p2, which, in contrast to the bulk rheology, formed a weak gel detectable by PTM. Overall, for both hSAF_{AAA} and hSAF_{QQQ}, G′ was greater than G″ at low temperatures, confirming gel formation. However, whereas hSAF_{AAA} showed a slight increase in gel strength with temperature, hSAF_{QQQ} showed a transition to a liquid state between 16 and 19° C., followed by a switch back to a gel state at higher temperatures.

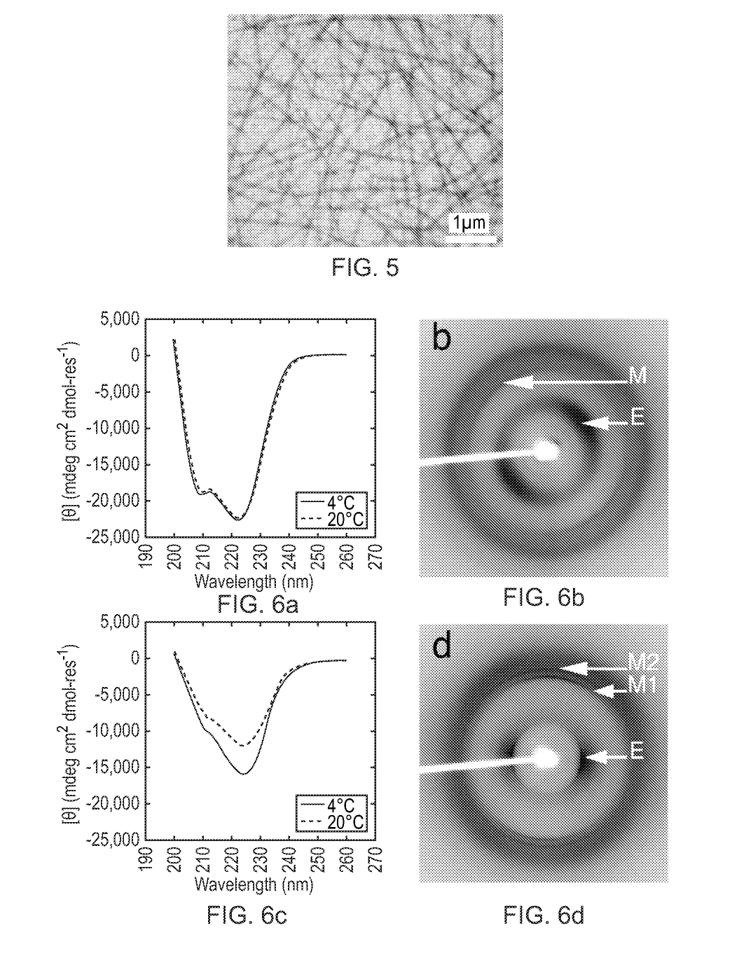

The formation of fibrils within the hydrogels was confirmed by low-temperature field emission scanning electron microscopy (FIG. 4B-E). For both hSAF_{AAA} and hSAF_{QQQ}, the samples prepared on ice showed interconnected fibres with polydisperse widths, but without uninterrupted networks (FIGS. 4B and 4C). Interestingly, on warming to room temperature, hSAF_{AAA} samples showed a homogeneous uninterrupted network of thinner fibers (FIG. 4E). The images for the control peptide, hSAF_{AAQ}, revealed no fibrous structures or networks (FIG. 4D), though unconnected fibres were visible by standard, negative-stain transmission electron microscopy of samples prepared at 20° C. (FIG. 5).

The peptide secondary structure and its packing in the fibril assemblies was probed by circular dichroism (CD) spectroscopy and x-ray fibre diffraction (XRD), respectively. CD spectra recorded at 4° C. and 20° C. for both hSAF_{AAA} and hSAF_{QQQ} were characteristic of α-helical structure (FIGS. 6a&c). That for the hSAF_{AAA} was the more intense and did not change upon heating to 20° C., while the spectrum for hSAF_{QQQ} lost intensity upon heating and demonstrated distortion due to light scattering (Papapostolou et al., 2008). These data are consistent with the gelation experiments described above.

XRD was performed on hSAF_{AAA} at 20° C. and on hSAF_{QQQ} at 4° C. (FIGS. 6B&D). In both cases, the diffraction patterns were similar to those presented for the other SAF systems (Papapostolou et al., 2007), although the unaligned fibres within the hSAF gels resulted in more-diffuse patterns with strong circular rings from water. hSAF_{AAA} and hSAF_{QQQ} gels both gave diffraction patterns with meridional reflections at 5.15 Å (M), corresponding to the 5.4 Å helical repeat of an α-helix supercoiled within a coiled coil.

The sharper meridional arc (M2) suggested some cross-β structure in the hSAF_{QQQ} sample (Blake & Serpell, 1996). The inventors posit that this is likely due to the high glutamine content of this sequence, which favours amyloid-like assemblies in other systems (Perutz et al., 1994; Sikorski & Atkins, 2005). However, cross-β structure normally gives a stronger signal in XRD; thus, the comparatively weak reflection in FIG. 6d, together with the predominantly α-helical CD spectrum (FIG. 6c), indicate only very small levels of β-structure in the hSAF_{QQQ} hydrogel.

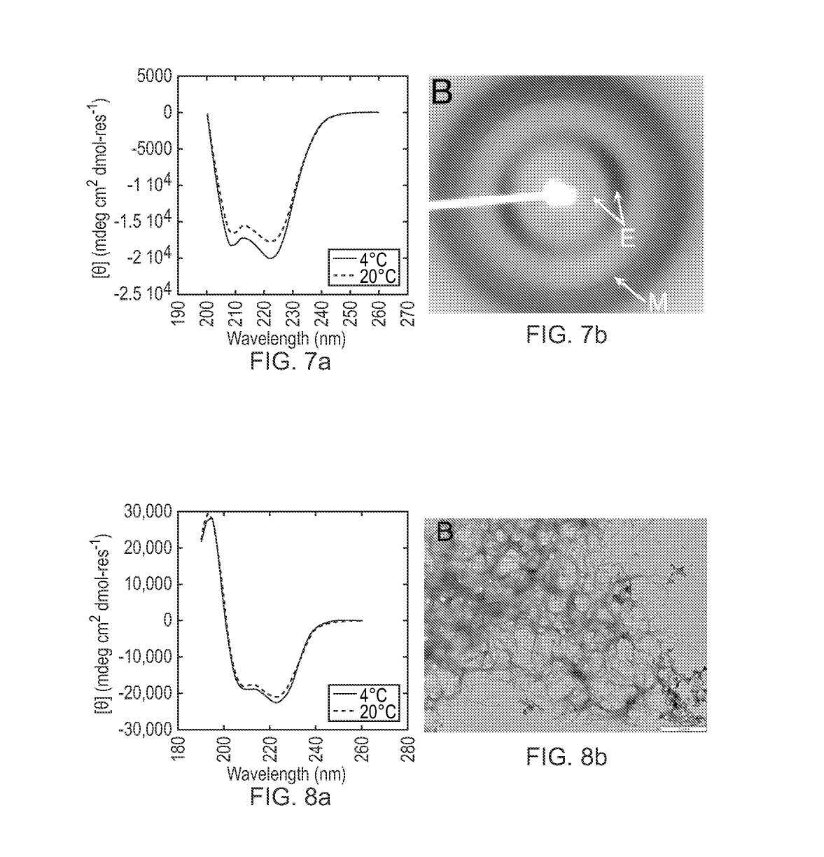

Regarding the structural organization within the α-helical fibrils of the hSAF_{AAA} and low-temperature hSAF_{QQQ} gels, the inventors have reported previously that for the standard, non-gelling SAFs the equatorial reflections in the XRD (FIGS. 6b&d) relate to the packing of the coiled coils on a hexagonal lattice (Papapostolou et al., 2007). Due to overlap of some the reflections in the XRD data for the hSAF gels, however, it was not possible to index these arcs completely. Nonetheless, by comparison with our foregoing studies, it was possible to assess the packing distances between coiled coils in the gels. In the standard SAFs, coiled coils are 18.2 Å apart (Papapostolou et al., 2007). From the new data for hSAF_{AAA} and hSAF_{QQQ} the corresponding separations were 17.3 Å and 21.5 Å, respectively. These spacings correlate with the changes to the sequences: for hSAF_{AAA} closer packing is expected because of the shorter alanine side chains; whereas, in hSAF_{QQQ} an increase might be expected because of (1) the replacement of predominantly alanine residues at b and c with the larger glutamine, and (2) the likely additional solvation of these hydrophilic residues. CD spectra and XRD patterns, consistent with these assertions were obtained for the hSAF_{AAQ} control fibres (FIG. 7). CD spectra recorded for hSAF_{AAQ} were typical of α-helical assemblies (FIG. 7A). Heating to 20° C. resulted in a slight loss of secondary structure (FIG. 7A). XRD of hSAF_{AAQ} had a meridional reflection at 5.15 Å consistent with α-helical assemblies (labelled M in FIG. 7B). Although several equatorial reflections were apparent (labelled E), they were insufficiently clear to calculate the packing distance between fibrils within the fibres.

To probe the utility of the hSAF_{AAA} gels as a substrate for cell growth, the inventors tested for peptide cytotoxicity and cell differentiation using rat adrenal pheochromocytoma (PC12) cells. First, however, the inventors had to further stabilize the fibril-fibril interactions and the resulting gels. This was because, though hSAF_{AAA} gels could be washed and soaked in both phosphate-buffered saline (PBS) and standard cell-culture media, they did not persist for sufficient time to allow sustained cell-culture experiments. To stabilize the gels, in each of the hSAF_{AAA} peptides the inventors replaced one of the surface-exposed alanine residues at an f position with the more hydrophobic tryptophan (Table 1). This also allowed easy quantification of peptide concentration. In all respects—spectroscopic, microscopic and gel formation—the hSAF_{AAA-W} combination behaved similarly to the parent peptides (FIG. 8). Moreover, the new peptides gelled at room temperature and the gels were stable in PBS and cell-culture media at 37° C. for more than two weeks, which permitted cell-biology studies as follows.

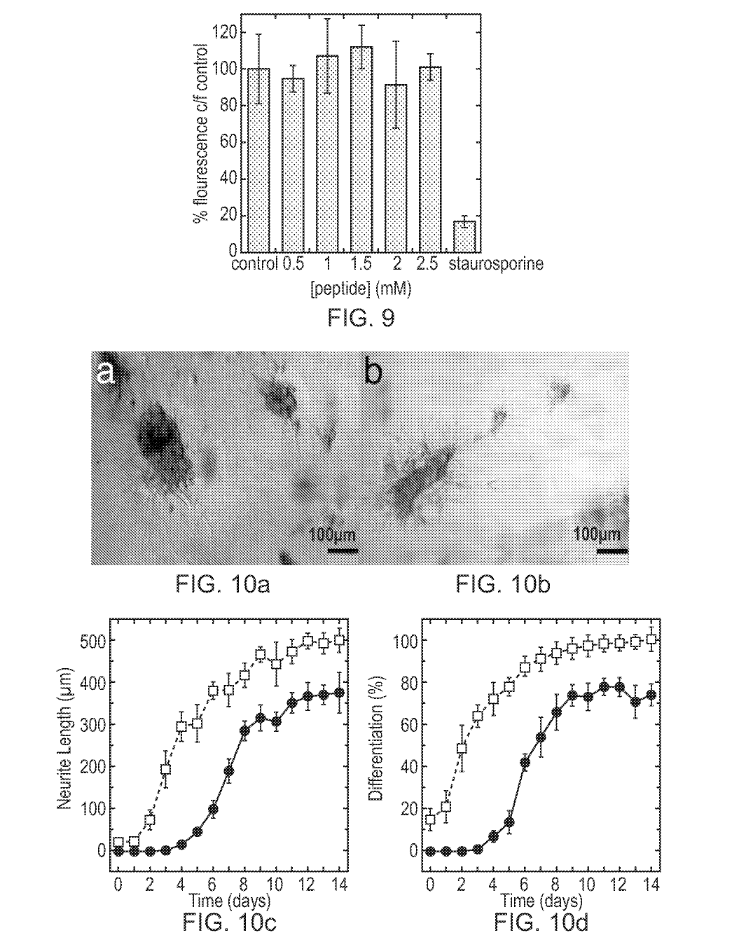

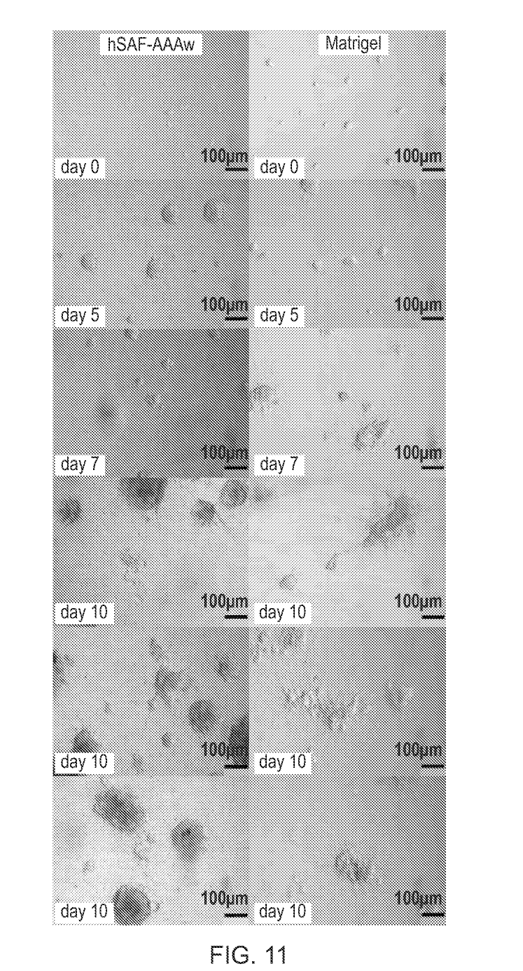

In Alamar Blue cell-proliferation assays (Hamid et al., 2004), PC12 cells seeded on collagen and then treated with increasing concentrations (0.5-2.5 mM, equivalent to 1.5-7.5 mg/ml, total peptide) of hSAF_{AAA-W} peptides and gels proliferated, and were statistically no different to controls without peptide. This was in contrast to similarly prepared cells treated with staurosporine, a known inducer of apoptosis, which died (FIG. 9). Moreover, PC12 cells seeded on hSAF_{AAA-W} gels (without collagen) could be induced to differentiate into neural cells using nerve growth factor at 100 ng/ml medium (Drubin et al., 1985), as judged by the presence of neurite projections from the cell bodies (FIG. 10A). As shown by phase-contrast microscopy (FIGS. 10A and 10B), the appearance of cells seeded on the hSAF_{AAA-W} gels was similar to those seeded on the widely used, but more-complex and ex vivo Matrigel substrate (Debnath et al., 2003). Despite also using NS-1 cells, which are believed not to form aggregates, many of the induced cells ingressed the gels clustered in three dimensions and both with hSAFs and Matrigel; i.e., achieving 3D cell cultures. N.B. multiple images from the first 10 days of these comparative cell-culture experiments are given in FIG. 11.

To compare cell differentiation within the hSAF and Matrigel substrates semi-quantitatively, the inventors followed neurite extension with time (FIG. 10C), and gauged overall differentiation in each culture (FIG. 10D). A cell was defined to have differentiated if it had axodendritic processes longer than 2-cell body diameters in length, i.e. processes longer than 20 μm (Todoroki et al., 2004). Though there was a lag in process growth and, consequently, cell differentiation in hSAF gels compared with Matrigel, on both counts the hSAF substrate performed at ˜75% of Matrigel by 10 days. In making this comparison, it is important to bear in mind that hSAF is a well-defined de novo substrate without any of natural structural proteins and associated cell-recognition motifs, or growth factors inherently present in Matrigel. Therefore, the performance of cells on hSAFs is particularly encouraging. In principle however, defined functionalities and additional factors could be engineered or added in known and controlled ways in future.

The hSAF peptides presented here gel at a peptide concentration of 1 mM (˜3 mg/ml) in each peptide; that is, they have >99% water content. Moreover, as shown above, changing the nature of the outer surfaces of the coiled coils—and, therefore, the inter-fibril interactions—allows temperature-responsive hydrogel properties to be engineered. This interesting and potentially useful behaviour warrants further comment. The hSAF_{QQQ} peptides, which have surface polar residues—i.e., glutamine residues at the f positions of the coiled-coil repeat that have amide side chains and hydrogen-bonding potential—assemble to weak gels at low temperature and melt on warming. This is consistent with the breaking of weak hydrogen-bonded cross-links between fibrils in a wet peptide gel. Whereas, hSAF_{AAA} peptides—which present only methyl side chains on their outer surfaces—form gels that become stronger on warming and are stable up to at least 95° C. This is consistent with hydrophobic cross-links between peptide fibrils. In contrast, the control peptides, hSAF_{AAQ}, in which the chemical symmetry of the outer surfaces is broken, do form fibres, but these do not form uninterrupted inter-fibril interactions and do not gel. The observations of gelation by the hSAF system are fully consistent with the initial design principles for modifying the fibril surfaces, and further demonstrate that the rational design of increasingly complex biomaterial systems is possible through different routes.

The demonstration that hSAFs support cell growth and differentiation is encouraging for the application of these gels as straightforward, chemically defined and engineerable scaffolds for cell culture and tissue engineering. The hSAF systems also carry the distinct advantage that they have two peptide components, and, therefore, gel only upon mixing. Thus, these new designs encompass unprecedented control, and represent an exciting addition to the available arsenal of biomaterials and gels. In addition to using hSAFs to form hydrogels and demonstrating that these gels are able to support growth and differentiation of PC12 cells in the presence of NGF, the inventors have also explored if the addition of cell-adhesion motifs would influence cell growth on these synthetic scaffolds. The inventors have described the functionalisation of SAFs using biorthogonal click chemistry (Mahmoud and Woolfson 2010), and shown that it is possible to modulate the display of gold nanoparticles and fluorophores on the surface of these fibers. A similar approach is used to add function to the hSAFs. The azide group is introduced into hSAF-p1 by replacing the terminal alanine with an azide-derivatized lysine in the fourth heptad repeat. This new peptide hSAF-p1N₃ readily co-assembles with hSAF-p2 to form fibres and gels similar to the parent (non-decorated) hSAF. Circular dichroism spectroscopy showed α-helical fibres with enhanced helicity of the azide-decorated fibrils. Transmission electron microscopy revealed long and floppy fibres with 15-25 nm diameter size. Scanning electron microscopy showed highly porous network of scaffolds. Microrheology studies showed gel strength similar to the parent hSAF-AAAw as described above.

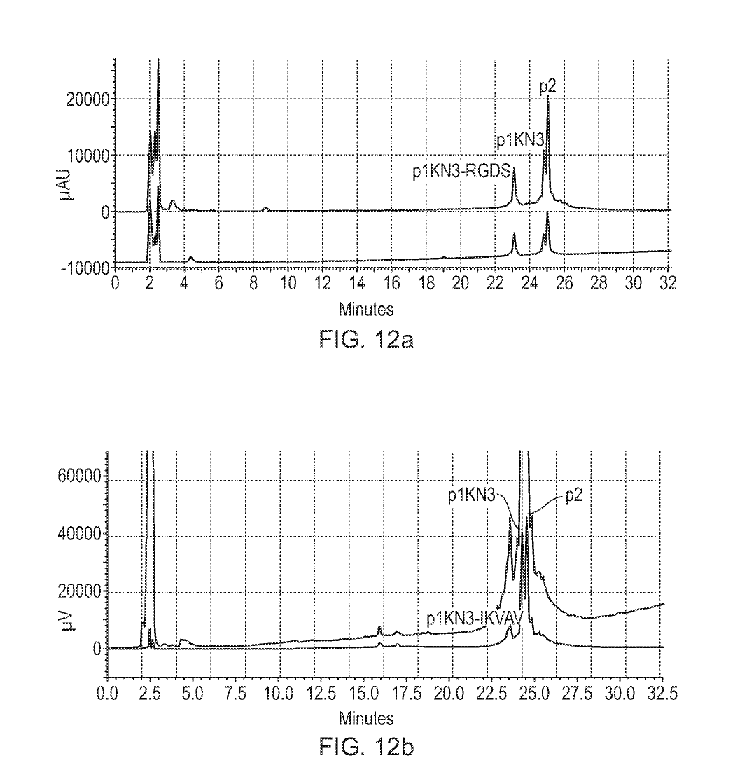

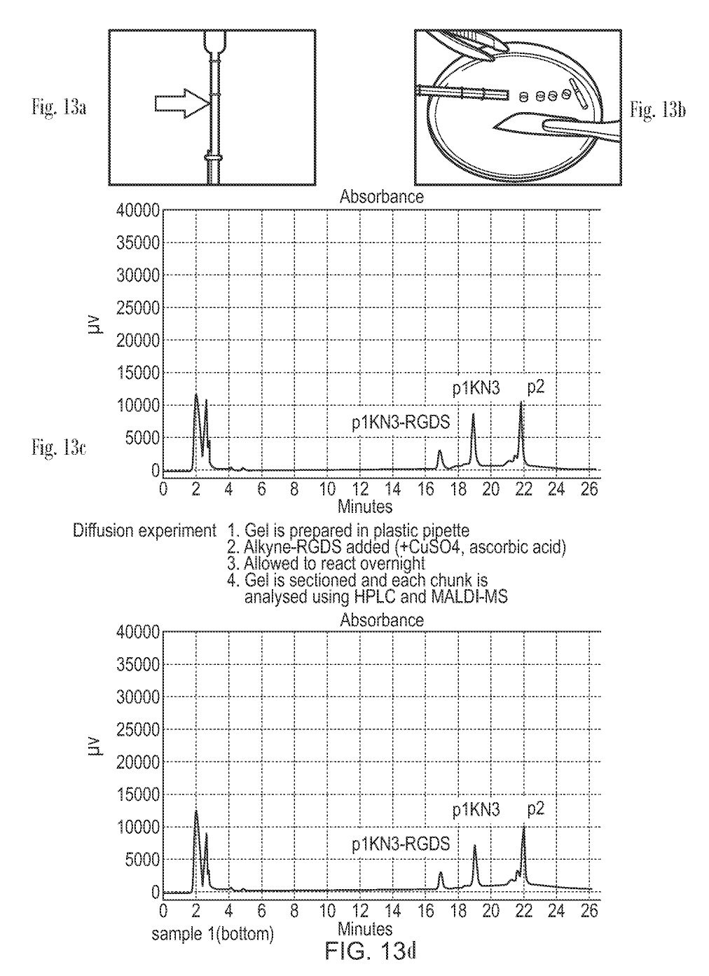

The cell-adhesion motif RGD was then integrated to the fibres via click chemistry. Alkyne was coupled to the N-terminus of the biomimetic peptide, with a spacer in between to facilitate access of the cells to this motif when displayed on the scaffold. Next, conditions for click chemistry were optimised by varying ratios of copper, ascorbic acid and alkyne. These conditions were optimised for the hSAF-p1N₃ peptide and subsequently for fibres and gels post-assembly. To confirm that the cyclo-addition of the biomimetic peptide has occurred, the decorated fibres were disassembled with 20% acetonitrile (Sigma) and 0.1% trifluoroacetic acid, TFA (Sigma), and analysed using high pressure liquid chromatography (HPLC) and matrix-assisted light desorption ionisation (MALDI) mass spectroscopy. It was found that 2 mM copper, 2 mM ascorbic acid and 2 mM alkyne-peptide were sufficient to give >90% conversion in 24 hours). When tested on fibres post-assembly, the resultant click product dropped to ˜30%, possibly because majority of the azide handles are buried inside the fibre and not available for functionalisation (FIG. 12). To test if the gels could be functionalised both on the surface and in the inner layers, the gel was prepared in a Pasteur pipette with a depth of 40 mm and internal diameter of 4 mm. Gels were functionalised with alkyne-RGDS under the same conditions used for fibre assembly, then sectioned at 5 mm intervals. HPLC analysis of the individual sections shows that the RGDS-functionalised peptides can be detected both in the top and lowermost layers, showing that the gels are permeable to the reagents used for functionalisation and can truly be functionalised in 3-D (FIG. 13).

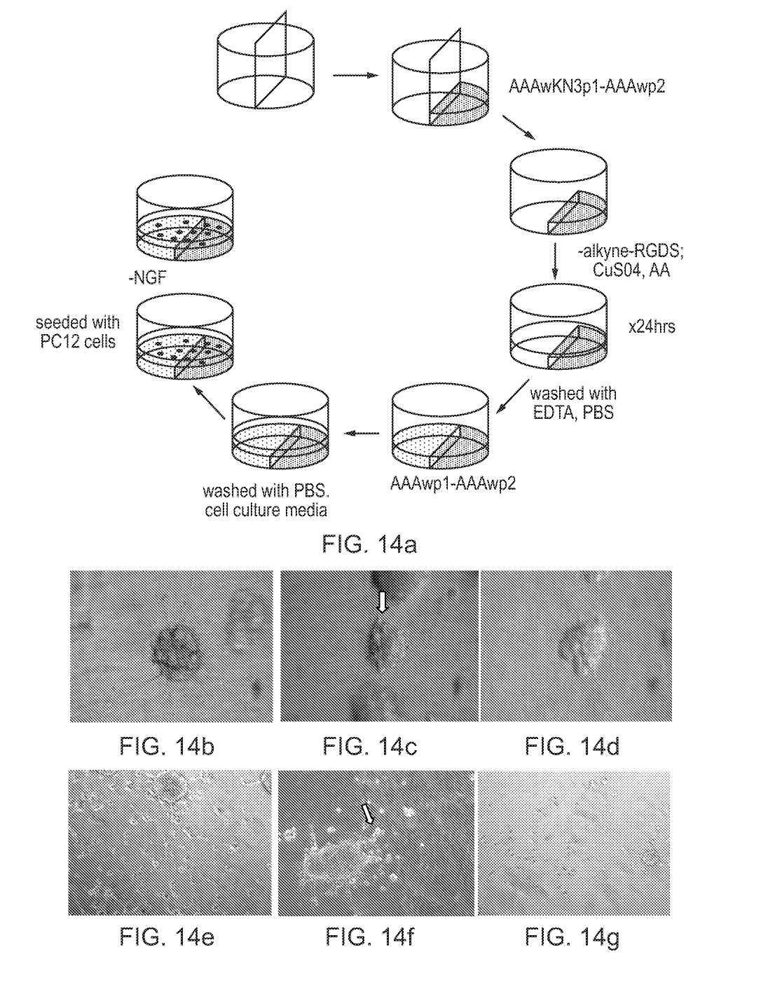

Next, the inventors tested various cell responses to these functionalised gels. The inventors used rat pheochromocytoma PC12 cells, rat primary hippocampal cells, and human dermal fibroblasts. To highlight the effect of the biomimetic peptide, the cells were maintained on either low-serum or serum-free media. PC12 cells were induced to differentiate by adding nerve growth factor (NGF) at 100 ng/ml. In a 24-well tissue culture plate, hydrogels were made with one side decorated with the RGDS peptide and the other side with plain hSAF (FIG. 14 and Methods). In brief, a pre-cut cover slip was placed perpendicular in the middle of the well to serve as boundary and the gels were assembled by mixing equimolar concentrations of hSAF-p1N₃ with hSAF-p2. After the gel had set, the cover slip was removed and the other half of the well was filled with plain hSAF with no azide group. The gels were allowed to set overnight at 37° C. which allowed two self-healing gels to merge. The next day, alkyne-RGDS in the presence of CuSO₄ and ascorbic acid were added as previously described. Excess copper was removed from the gels by extensively washing with 10 mM EDTA, at least 10 times. Cells were then seeded on them at concentration of 10,000/ml of media. The inventors found that the cell behaviour was affected by the presence of the RGDS motif. PC12 cells grown on decorated gel differentiated 2 days ahead of the control and their neurites were significantly longer (FIGS. 14B and D).

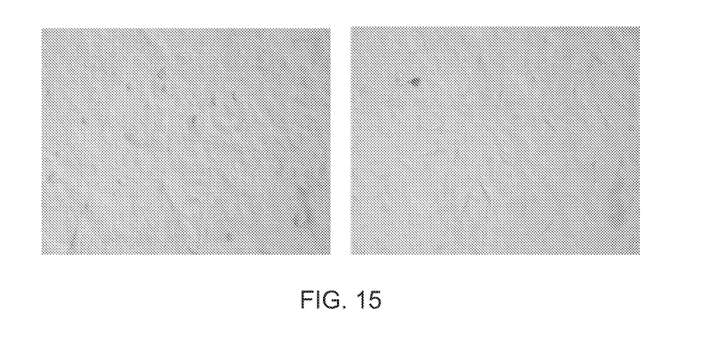

The hippocampal cells on functionalised gel likewise showed early neurite formation (FIGS. 14E and 14G). Though RGDS does not directly influence neurite growth, it promotes attachment of cells to the scaffold, thus these cells should be able to differentiate and sprout neurites in a shorter time period than cells grown on non-functionalised scaffolds. The fibroblasts on functionalised gel had stretched and spindle-shaped morphologies (FIG. 15).

An important factor influencing cell behaviour in the extracellular matrix is gradients of growth factors which cause the cell to polarise, migrate and organise into 3D structure. Interestingly, those cells that landed on the interface between the functionalised and non-functionalised gel showed significant outgrowth of axo-dendritic processes towards the side with RGDS decoration (FIGS. 14C and 14F).

All documents mentioned above are hereby incorporated by reference.

### REFERENCES

Aggeli, A. et al. Nature 386, 259-262 (1997).
Cicuta & Donald Soft Matter 3 1449 (2007).
Dawson et al. Science 266: 776-779 (1994).
Debnath, J., Muthuswamy, S. K. & Brugge, J. S. Methods 30, 256-268 (2003).
Dong, H., Paramonov, S. E. & Hartgerink, J. D. J. Am. Chem. Soc. 130, 13691-13695 (2008).
Drubin, D. G., Feinstein, S. C., Shooter, E. M. & Kirschner, M. W. J. Cell Biol. 101, 1799-1807 (1985).
Goddard-Borger and Stick, Org. Lett., 9, 3797-3800 (2007).
Gribbon, C. et al. Biochemistry 47, 10365-10371 (2008).
Hamid, R., Rotshteyn, Y., Rabadi, L., Parikh, R. & Bullock, P. Toxicol. Vitro 18, 703-710 (2004).
Hartgerink, J. D., Beniash, E. & Stupp, S. I. Science 294, 1684-1688 (2001).
Hasnain & Donald Phys. Rev. E 73, 031901 (2006).
Houghton et al. Eur. Phys. J. E 25, 119 (2008)
Kope\check{c}ek, J. & Yang, J. Y. Acta Biomater 5, 805-816 (2009).
Larsen & Furst Phys. Rev. Lett. 100, 146001 (2008).
Makin, O, S., Sikorski, P. & Serpell, L. C. J. Appl. Crystallogr. 40, 966-972 (2007).
Mason et al. J. Opt. Soc. Am. 14, 139 (1997).
Mason Rheol. Acta. 39, 371 (2000).
Pandya, M. J. et al. Biochemistry 39, 8728-8734 (2000).
Papagiannopoulos Faraday Discus. DOI: 10.1039/b714864j (2008).
Papapostolou, D. et al. Proc. Natl. Acad. Sci. USA 104, 10853-8 (2007).
Papapostolou, D., Bromley, E. H., Bano, C. & Woolfson, D. N. 130, 5124-30 (2008).
Paramonov, S., Gauba, V. & Hartgerink, J. Macromolecules 38, 7555-7561 (2005).
Perutz, M. F., Johnson, T., Suzuki, M. & Finch, J. T. Proc. Natl. Acad. Sci. USA 91, 5355-5358 (1994).
Petka, W. A., Harden, J. L., McGrath, K. P., Wirtz, D. & Tirrell, D. A. Science 281, 389-392 (1998).
Pochan, D. J. et al. J. Am. Chem. Soc. 125, 11802-11803 (2003).
Potekhin, S. A. et al. Chem. Biol. 8, 1025-32 (2001).
Ryadnov, M. G. & Woolfson, D. N. Nature Mater. 2, 329-32 (2003).
Savin & Doyle, Biophys J. 88, 623 (2005).
Schneider, J. P. et al. J. Am. Chem. Soc. 124, 15030-15037 (2002).
Sikorski, P. & Atkins, E. Biomacromolecules 6, 425-432 (2005).
Todoroki, S., Morooka, H., Yamaguchi, M., Tsujita, T. & Sumikawa, K. Anesth. Analg. 99, 828-832 (2004).
Ulijn, R. V. & Smith, A. M. Chem. Soc. Rev. 37, 664-675 (2008).
Wang, C., Stewart, R. J. & Kopecek, J. Nature 397, 417-420 (1999).
Winn, M. D. J. Synchrot. Radiat. 10, 23-25 (2003).
Woolfson, D. N. & Ryadnov, M. G. Curr. Opin. Chem. Biol. 10, 559-567 (2006).
Woolfson and Mahmoud, Chem. Soc. Rev. 39, 3464-3479 (2010).Zhang, S. G., Holmes, T., Lockshin, C. & Rich, A. Proc. Natl. Acad. Sci. USA 90, 3334-3338 (1993).
Zimenkov, Y., Conticello, V. P., Guo, L. & Thiyagarajan, P. Tetrahedron 60, 7237-7246 (2004).

We claim:

1. A protein fibril comprising a plurality of first peptide monomer units arranged in a first strand and a plurality of second peptide monomer units arranged in a second strand in which said first and second strands form an overlapping staggered heterodimer coiled-coil structure, and wherein the amino acid residues on exposed surfaces of said first and second strands enable said protein fibril to interact with another protein fibril in a plurality of non-parallel orientations.

2. A protein fibril according to claim 1, wherein the amino acid residues on the exposed surfaces of said first and second strands enable said protein fibril to interact with said another protein fibril through hydrophobic interactions.

3. A protein structure according to claim 1, wherein the amino acid residues on the exposed surfaces of said first and second strands enable said protein fibril to interact with said another protein fibril through H-bond interactions.

4. A protein structure according to claim 1, wherein the amino acid residues on the exposed surfaces of said first and second strands enable said protein fibril to interact with said another protein fibril through ionic interactions.

5. A protein fibril according to claim 1, further comprising a hub and a plurality of peptide monomer units each being attached at one end thereof to the hub, wherein free ends of at least 2 peptide monomer units are N-termini or C-termini, and each of the at least 2 peptide monomer units is capable of interacting with said first or second strand.

6. A protein fibril according to claim 1 in which the peptide monomer units comprise a repeating structural unit.

7. A protein fibril according to claim 6 in which the repeating structural unit comprises a heptad repeat motif (abcdefg).

8. A protein fibril according to claim 7 having amino acid residues with hydrophobic side chains at positions b, c and f so as to form hydrophobic interaction on the exposed surfaces of said first and/or second strand.

9. A protein fibril according to claim 8, wherein said amino acid residues with hydrophobic side chains are alanine and/or tryptophan.

10. A protein fibril according to claim 9 having alanine at positions b, c and f.

11. A protein fibril according to claim 9 having alanine at positions b and c, and tryptophan at position f.

12. A protein fibril according to claim 7 having amino acid residues with uncharged polar side chains at positions b, c and f so as to form H-bond interaction on the exposed surfaces of said first and/or second strand.

13. A protein fibril according to claim 12, wherein said amino acid residues with uncharged polar side chains are glutamine.

14. A protein fibril according to claim 10 in which the first and second peptide monomer units have the sequences:
(hSAF_{AAA} p1; SEQ ID NO: 2)
a) KIAALKAKIAALKAEIAALEAENAALEA
and
(hSAF_{AAA} p2; SEQ ID NO: 3)
b) KIAALKAKNAALKAEIAALEAEIAALEA
respectively.

15. A protein fibril according to claim 11 in which the first and second peptide monomer units have the sequences:
(hSAF_{AAA-W} p1; SEQ ID NO: 4)
a) KIAALKAKIAALKAEIAALEWENAALEA
and
(hSAF_{AAA-W} p2; SEQ ID NO: 5)
b) KIAALKAKNAALKAEIAALEWEIAALEA
respectively.

16. A protein fibril according to claim 13 in which the first and second peptide monomer units have the sequences:
(hSAF_{QQQ} p1; SEQ ID NO: 6)
a) KIQQLKQKIQQLKQEIQQLEQENQQLEQ
and
(hSAF_{QQQ} p2; SEQ ID NO: 7)
b) KIQQLKQKNQQLKQEIQQLEQEIQQLEQ
respectively.

17. A protein fibril according to claim 6 in which the repeating structural unit comprises a hendecad repeat motif (abcdefghijk).

18. A protein fibril according to claim 1 in which at least one amino acid residue is derivatised.

19. A protein fibril according to claim 18, wherein derivatisation is achieved using click chemistry.

20. A protein fibril according to claim 18, wherein the at least one amino acid residue is derivatised with a click group.

21. A protein fibril according to claim 20, wherein the click group is selected from a group consisting of azide, alkyne, thiol and alkene groups.

22. A protein fibril according to claim 18, wherein said at least one amino acid residue is derivatised with a macromolecule.

23. A protein fibril according to claim 22, wherein said macromolecule is selected from the group consisting of extracellular matrix components, small peptides, cytokines and hormones.

24. A protein fibril according to claim 22 or 23, wherein said macromolecule is attached to the at least one amino acid residue through an azide-alkyne or a thiol-ene unit.

25. A peptide monomer unit for use in preparing a protein fibril according to any preceding claim, the peptide monomer unit having an amino acid sequence selected from:
(hSAF_{AAA} p1; SEQ ID NO: 2)
a) KIAALKAKIAALKAEIAALEAENAALEA;
(hSAF_{AAA} p2; SEQ ID NO: 3)
b) KIAALKAKNAALKAEIAALEAEIAALEA;
(hSAF_{AAA-W} p1; SEQ ID NO: 4)
c) KIAALKAKIAALKAEIAALEWENAALEA;
(hSAF_{AAA-W} p2; SEQ ID NO: 5)
d) KIAALKAKNAALKAEIAALEWEIAALEA;
(hSAF_{QQQ} p1; SEQ ID NO: 6)
e) KIQQLKQKIQQLKQEIQQLEQENQQLEQ
and
(hSAF_{QQQ} p2; SEQ ID NO: 7)
f) KIQQLKQKNQQLKQEIQQLEQEIQQLEQ.

26. A peptide monomer unit according to claim 25, wherein one amino acid residue in the peptide monomer unit is substituted with a derivatised amino acid residue.

27. A peptide monomer unit according to claim 26, wherein substitution occurs at the C-terminus of the peptide monomer unit.

28. A peptide monomer unit according to claim 26, wherein the at least one amino acid residue is substituted by a derivatised lysine residue.

29. A peptide monomer unit according to claim 25 in which at least one amino acid residue is derivatised.

30. A peptide monomer unit according to claim 29, wherein derivatisation is achieved using click chemistry.

31. A peptide monomer unit according to claim 30, wherein the at least one amino acid residue is derivatised with a click group.

32. A peptide monomer unit according to claim 31, wherein the click group is selected from a group consisting of azide, alkyne, thiol and alkene groups.

33. A peptide monomer unit according to claim 29, wherein said at least one amino acid residue is derivatised with a macromolecule.

34. A peptide monomer unit according to claim 33, wherein said macromolecule is selected from the group consisting of extracellular matrix components, small peptides, cytokines and hormones.

35. A peptide monomer according to claim 33, wherein said macromolecule is attached to the at least one amino acid residue through an azide-alkyne or a thiol-ene unit.

36. A method of producing a protein fibril, the method comprising providing first and second peptide monomer units that associate to form a protein structure according to claim 1, and mixing said first and second peptide monomer units together.

37. A kit for making a protein fibril, the kit comprising first and second peptide monomer units that associate to form a protein fibril according to claim 1.

38. A bundle of protein fibrils produced by an association of a plurality of protein fibrils according to claim 1, wherein at least one protein fibril of said bundle associates with another protein fibril of said bundle in a plurality of non-parallel orientations.

39. A three dimensional matrix structure produced by association of protein fibrils according to claim 1, or of bundles of protein fibrils produced by association of a plurality of said protein fibrils, or of a mixture of the above.

40. A three dimensional matrix structure according to claim 39, wherein the three dimensional matrix is a hydrogel.

41. A three dimensional matrix structure according to claim 40, wherein the hydrogel has a water content of at least 85%.

42. A three dimensional matrix structure according to claim 40, wherein the hydrogel is formed at a low temperature and strengthens on warming.

43. A three dimensional matrix structure according to claim 40, wherein the gel is formed at a low temperature, melts on warming up to 35° C. and reforms at a temperature above 35° C.

44. A three dimensional matrix structure according to claim 39 further comprising macromolecules.

45. A three dimensional matrix structure according to claim 44, wherein said macromolecules are selected from extracellular matrix components, small peptides, cytokines and hormones.

46. A three dimensional matrix structure according to claim 45 for use in cell growth.

47. A three dimensional matrix structure according to claim 45 for use in tissue engineering.

48. A method of making a three dimensional matrix according to claim 39, said method comprising a) providing first and second peptide monomer units which associate to form a protein fibril, and mixing said first and second peptide monomer units together; or b) providing a mixture of protein fibrils; and the accumulation and assembling of such protein fibrils or bundles of protein fibrils forming a three dimensional matrix, wherein said fibril or fibrils comprise a plurality of first peptide monomer units arranged in a first strand and a plurality of second peptide monomer units arranged in a second strand in which said first and second strands form an overlapping staggered heterodimer coiled-coil structure, and wherein the amino acid residues on exposed surfaces of said first and second strands enable said protein fibril to interact with another protein fibril in a plurality of non-parallel orientations.

49. A method of claim 48 further comprising a step of addition macromolecules to the mixture.

50. A method of claim 48, wherein the method is carried out in the presence of an aqueous solution and the resulting three dimensional matrix is a hydrogel.

51. A kit for making a three dimensional matrix, the kit comprising
a) first and second peptide monomer units which, upon mixing, associate to form a protein fibril according to claim 1; or
b) a mixture of protein fibrils according to claim 1.

52. A kit of claim 51 further comprising macromolecules.

53. A self-assembling peptide monomer unit, wherein the peptide monomer unit is derivatised with a click group.

54. A protein fibril comprising a plurality of first self-assembling peptide monomer units arranged in a first strand and a plurality of second self-assembling peptide monomer units arranged in a second strand in which said first and second strands form an overlapping staggered heterodimer coiled-coil structure, wherein at least one self-assembling peptide monomer unit is derivatised with a click group.

55. A self-assembling peptide monomer unit according to claim 53, wherein the click group is a group selected from a group consisting of azide, alkene, alkyne and thiol groups.

56. A protein fibril according to claim 54, wherein the click group is a group selected from a group consisting of azide, alkene, alkyne and thiol groups.

57. A method of derivatising a self-assembling peptide monomer unit using click chemistry comprising incorporating a first click group into the self-assembling peptide monomer unit, reacting the resultant peptide monomer unit with a molecule having a second click group which is complementary to the first click group, such that the monomer unit is attached to the molecule through the first and second click groups.

58. A method of derivatising a protein fibril using click chemistry comprising incorporating a first click group into at least a first and/or at least a second self-assembling peptide monomer unit, mixing the at least first and/or the at least second self-assembling peptide monomer unit having the first click group with a plurality of first and second self-assembling peptide monomer units together, reacting the resultant protein fibril with a molecule having a second click group which is complementary to the first click group, such that the protein fibril is attached to the molecule through the first and second click groups.
